SEQUENCE LISTING (42 sequences)

SEQ ID NO: 1 - DNA, 21290 bp, SARS-CoV2
  ORF1ab
atggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaa

SEQ ID NO: 2 - DNA, 1911 bp, Artificial Sequence
  Reo
atggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagaattctgcttgcaagaactggttcagtagcttaagccactttgtgatccaccttaacagccacggcttccctcccgaggtggaggagcaggccgccggcaccctgcccatgagctgcgcccaggagagcggcatggatagacaccctgctgcttgcgccagcgccaggatcaacgtccggcgcgccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 3 - DNA, 1260 bp, SARS-CoV2
  N
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 4 - DNA, 6 bp, SARS-CoV2
  TRS
acgaac

SEQ ID NO: 5 - DNA, 129 bp, SARS-CoV2
  ORF8
acagtttcctgtttaccttttacaattaattgccaggaacctaaattgggtagtcttgtagtgcgttgttcgttctatgaagactttttagagtatcatgacgttcgtgttgttttagatttcatctaa

SEQ ID NO: 6 - DNA, 8 bp, SARS-CoV2
  RS
aaactaaa

SEQ ID NO: 7 - DNA, 1273 bp, Artificial Sequence
  fragment 1
gagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaac

SEQ ID NO: 8 - DNA, 2423 bp, Artificial Sequence
  fragment 2
acgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtg

SEQ ID NO: 9 - DNA, 2470 bp, Artificial Sequence
  fragment 3
caaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaagagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttg

SEQ ID NO: 10 - DNA, 2131 bp, Artificial Sequence
  fragment 4
catagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtagtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtc

SEQ ID NO: 11 - DNA, 2264 bp, Artificial Sequence
  fragment 5
gcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgc

SEQ ID NO: 12 - DNA, 2802 bp, Artificial Sequence
  fragment 6
gtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacatta

SEQ ID NO: 13 - DNA, 3516 bp, Artificial Sequence
  fragment 7
aggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagtt

SEQ ID NO: 14 - DNA, 4279 bp, Artificial Sequence
  fragment 8
cttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaacactggcgcgccacagtttcctgttta

SEQ ID NO: 15 - DNA, 2107 bp, Artificial Sequence
  fragment 9
tgttaacaactaacactggcgcgccacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagaattctgcttgcaagaactggttcagtagcttaagccactttgtgatccaccttaacagccacggcttccctcccgaggtggaggagcaggccgccggcaccctgcccatgagctgcgcccaggagagcggcatggatagacaccctgctgcttgcgccagcgccaggatcaacgtccggcgcgccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggatccacagtttcctgtttacctttta

SEQ ID NO: 16 - DNA, 10963 bp, Artificial Sequence
  fragment 10
acgagttcttctgaggatccacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaagggtcggcatggcatctccacctcctcgcggtccgacctgggcatccgaaggaggacgtcgtccactcggatggctaagggagagctcggatcgatccgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcgagctctgtacatgtccggcggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattaatacgactcactatagggtcagtgctcgagtaagttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatc

SEQ ID NO: 17 - DNA, 30 bp, Artificial Sequence
  fragment 1 forward primer
gagctggtggccatagttacggcgccgatc

SEQ ID NO: 18 - DNA, 28 bp, Artificial Sequence
  fragment 1 reverse primer
gtttcaagagtgcgggagaaaattgatc

SEQ ID NO: 19 - DNA, 30 bp, Artificial Sequence
  fragment 2 forward primer
acgatcaattttctcccgcactcttgaaac

SEQ ID NO: 20 - DNA, 30 bp, Artificial Sequence
  fragment 2 reverse primer
cacttttacactttttaagcactgtctttg

SEQ ID NO: 21 - DNA, 30 bp, Artificial Sequence
  fragment 3 forward primer
caaagacagtgcttaaaaagtgtaaaagtg

SEQ ID NO: 22 - DNA, 30 bp, Artificial Sequence
  fragment 3 reverse primer
caaacacggtttaaacaccgtgtaactatg

SEQ ID NO: 23 - DNA, 30 bp, Artificial Sequence
  fragment 4 forward primer
catagttacacggtgtttaaaccgtgtttg

SEQ ID NO: 24 - DNA, 30 bp, Artificial Sequence
  fragment 4 reverse primer
gacaaaacccacttctcttgttatgactgc

SEQ ID NO: 25 - DNA, 30 bp, Artificial Sequence
  fragment 5 forward primer
gcagtcataacaagagaagtgggttttgtc

SEQ ID NO: 26 - DNA, 30 bp, Artificial Sequence
  fragment 5 reverse primer
gcattttcatacaaaaaaaagaacaaagac

SEQ ID NO: 27 - DNA, 30 bp, Artificial Sequence
  fragment 6 forward primer
gtctttgttcttttttttgtatgaaaatgc

SEQ ID NO: 28 - DNA, 30 bp, Artificial Sequence
  fragment 6 reverse primer
taatgtgtcacaattaccttcatcaaaatg

SEQ ID NO: 29 - DNA, 37 bp, Artificial Sequence
  fragment 7 forward primer
aggcattttgatgaaggtaattgtgacacattaaaag

SEQ ID NO: 30 - DNA, 30 bp, Artificial Sequence
  fragment 7 reverse primer
aactatatctgctgtcgtctcaggcaatgc

SEQ ID NO: 31 - DNA, 30 bp, Artificial Sequence
  fragment 8 forward primer
cttttgtactgtaaatgcattgcctgagac

SEQ ID NO: 32 - DNA, 30 bp, Artificial Sequence
  fragment 8 reverse primer
taaacaggaaactgtggcgcgccagtgtta

SEQ ID NO: 33 - DNA, 30 bp, Artificial Sequence
  fragment 9 forward primer
tgttaacaactaacactggcgcgccacagt

SEQ ID NO: 34 - DNA, 30 bp, Artificial Sequence
  fragment 9 reverse primer
taaaaggtaaacaggaaactgtggatcctc

SEQ ID NO: 35 - DNA, 30 bp, Artificial Sequence
  fragment 10 forward primer
gatcggcgccgtaactatggccaccagctc

SEQ ID NO: 36 - DNA, 30 bp, Artificial Sequence
  fragment 10 reverse primer
acgagttcttctgaggatccacagtttcct

SEQ ID NO: 37 - DNA, 33886 bp, Artificial Sequence
  pBAC-SCoV2-Rep-Reo
ggccgcttctatagtgtcacctaaatactagtgactccagcgtaactggactggccacagttaggccgcaaatgtaatcacactggctcaccttcgggtgggcctttctgcgttcgcgagtggacccgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggaggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatggtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgcttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgcggccgatctagcagaaagtcaaaagcctccgaccggaggcttttgacttctgtcacctaggttacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcagattaaaacgaaaggcccagtctttcgactgagcctttcgttttatttgaccatgttggtatgatttaaattaatacgactcactatagggtcagtgctcgagtaagttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaacactggcgcgccacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagaattctgcttgcaagaactggttcagtagcttaagccactttgtgatccaccttaacagccacggcttccctcccgaggtggaggagcaggccgccggcaccctgcccatgagctgcgcccaggagagcggcatggatagacaccctgctgcttgcgccagcgccaggatcaacgtccggcgcgccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggatccacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaagggtcggcatggcatctccacctcctcgcggtccgacctgggcatccgaaggaggacgtcgtccactcggatggctaagggagagctcggatcgatccgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagctcgagctctgtacatgtccgc

SEQ ID NO: 38 - DNA, 792 bp, Artificial Sequence
  probe for N
accacgttcccgaaggtgtgacttccatgccaatgcgcgacattccgaagaacgctgaagcgctgggggcaaattgtgcaatttgcggccaatgtttgtaatcagttccttgtctgattagttcctggtccccaaaatttccttgggtttgttctggaccacgtctgccgaaagcttgtgttacattgtatgctttagtggcagtacgtttttgccgaggcttcttagaagcctcagcagcagatttcttagtgacagtttggccttgttgttgttggcctttaccagacattttgctctcaagctggttcaatctgtcaagcagcagcaaagcaagagcagcatcaccgccattgccagccattctagcaggagaagttcccctactgctgcctggagttgaatttcttgaactgttgcgactacgtgatgaggaacgagaagaggcttgactgccgcctctgctcccttctgcgtagaagccttttggcaatgttgttccttgaggaagttgtagcacgattgcagcattgttagcaggattgcgggtgccaatgtgatcttttggtgtattcaaggctccctcagttgcaacccatatgatgccgtctttgttagcaccatagggaagtccagcttctggcccagttcctaggtagtagaaataccatcttggactgagatctttcattttaccgtcaccaccacgaattcgtctggtagctcttcggtagtagccaatttggtcatctggactgctattggtgttaattggaacgccttgtcctcgag

SEQ ID NO: 39 - DNA, 24 bp, Artificial Sequence
  N forward primer
cgatctcttgtagatctgttctct

SEQ ID NO: 40 - DNA, 21 bp, Artificial Sequence
  N reverse primer
gtcttccttgccatgttgagt

SEQ ID NO: 41 - DNA, 19 bp, Artificial Sequence
  GAPDH forward primer
gaaggtgaaggtcggagtc

SEQ ID NO: 42 - DNA, 20 bp, Artificial Sequence
  GAPDH reverse primer
gaagatggtgatgggatttc

(19) United States
(12) Patent Application Publication
(10) Pub. No.: US 2022/0372448 A1
(43) Pub. Date: Nov. 24, 2022

(54) CELL LINE AND PRODUCING METHOD THEREOF

(71) Applicant: University of Yamanashi, Kofu-shi, Yamanashi (JP)

(72) Inventors: Kohji MORIISHI, Yamanashi (JP); Tomohisa TANAKA, Yamanashi (JP)

(73) Assignee: University of Yamanashi, Kofu-shi, Yamanashi (JP)

(21) Appl. No.: 17/744,324

(22) Filed: May 13, 2022

(30) Foreign Application Priority Data
May 18, 2021 (JP) 2021-084127
May 10, 2022 (JP) 2022-077289

(51) Int. Cl. C12N 5/10; C07K 14/08; C12N 15/86

(52) U.S. Cl. CPC C12N 5/10; C07K 14/08; C12N 15/86; C12N 2770/20041

(57) ABSTRACT

A cell line of Accession No. NITE P-03458. A producing method of a cell line includes: a transfection step of transfecting a vector into a cell, where the vector includes a nucleic acid sequence encoding a nonstructural protein of a coronavirus, a reporter protein including a drug-resistant protein, and a structural protein of the coronavirus; and a selection step of performing selection on the cell obtained in the transfection step using a drug.

### CROSS-REFERENCE TO RELATED APPLICATIONS

The present application claims priority under 35 U.S.C. § 119 to Japanese Patent Application No. 2021-084127, filed May 18, 2021, and Japanese Patent Application No. 2022-077289, filed May 10, 2022. The contents of which are incorporated herein by reference in their entirety.

### BACKGROUND OF THE INVENTION

### Field of the Invention

The present disclosure relates to a cell line and a producing method thereof.

### Description of the Related Art

In response to the global spread of infection with the new coronavirus (COVID-19), there is a demand for development of a virus-inhibiting drug that suppresses proliferation of this virus. In general, development of a virus-inhibiting drug has a high hurdle and requires a large-scale screening experiment using, for example, a compound library. For the new coronavirus (COVID-19), several cell lines are available for experiments of viral infection. Currently, therefore, the screening experiment as mentioned above is being conducted using the infectious virus.

However, since the new coronavirus (COVID-19) having infectivity can be handled only in BSL3 facilities, it is necessary to convey potentially virus-contaminated equipment into the BSL3 facilities. For this reason, the current drug discovery research for the new coronavirus (COVID-19) is often performed under restrictions.

In view thereof, a system designed to express replicon RNA of the new coronavirus (COVID-19) has been developed (Scientific Reports 2021 11:2229; Antiviral Research 2021 185:104974; and mBio 2021 12:e02754-20).

However, in this system, replicon RNA is transiently expressed, and a cell line that stably expresses replicon RNA cannot be obtained. Therefore, a replicon-expressing vector is necessary to be synthesized every time and transfected into cells. The size of the replicon-expressing vector is huge to cause unstability upon preparation, which further causes variation in transfection efficiency (BioTechniques 2003 35:796-807).

Such restrictions make it difficult to realize a large-scale screening experiment like drug discovery research.

As a system designed to express replicon RNA of hepatitis C virus (HCV), reported is a cell line that is produced by a method of transfecting the replicon RNA of HCV and stably expresses the replicon RNA of HCV (Science 1999 285:110-3).

According to Scientific Reports 2021 11:2229, the replicon RNA of the coronavirus is transiently expressed in culture cells by the same method as described in Science 1999 285:110-3; i.e., by the method of transfecting the replicon RNA of the coronavirus. However, this method still cannot produce a cell line that stably expresses the replicon RNA of the coronavirus.

Under such circumstances, there is a strong demand for rapidly providing a cell that stably expresses replicon RNA of a coronavirus and enables highly sensitive and highly reproducible screening for an anti-coronavirus therapeutic drug.

### SUMMARY OF THE INVENTION

The present disclosure aims to solve the above existing problems in the art and achieve the following object. Specifically, the present disclosure has an object to provide a cell that stably expresses replicon RNA of a coronavirus and enables highly sensitive and highly reproducible screening for an anti-coronavirus therapeutic drug.

The present inventors conducted intensive studies for solving the above problems. As a result, the present inventors have found that it is possible to provide a cell that stably expresses replicon RNA of a coronavirus and enables highly sensitive and highly reproducible screening for an anti-coronavirus therapeutic drug, by a producing method of a cell line including: a transfection step of transfecting a vector into a cell, where the vector includes a nucleic acid sequence encoding a nonstructural protein of the coronavirus, a reporter protein including a drug-resistant protein, and a structural protein of the coronavirus; and a selection step of performing selection on the cell obtained in the transfection step using a drug.

The present disclosure is based on the above finding obtained by the present inventors and means for solving the problems are as follows.

<1> A cell line, which is of Accession No. NITE P-03458.

<2> A producing method of a cell line, the producing method including:

a transfection step of transfecting a vector into a cell, where the vector includes a nucleic acid sequence encoding a nonstructural protein of a coronavirus, a reporter protein including a drug-resistant protein, and a structural protein of the coronavirus; and

a selection step of performing selection on the cell obtained in the transfection step using a drug.

<3> A vector, including:

a nucleic acid sequence encoding a nonstructural protein of a coronavirus, a reporter protein including a drug-resistant protein, and a structural protein of the coronavirus.

<4> An introduced cell, including:

the vector according to <3> above, which is introduced into the introduced cell.

<5> A screening method for an anti-coronavirus therapeutic drug, the screening method including:

using the introduced cell according to <4> above.

<6> A screening kit for an anti-coronavirus therapeutic drug, the screening kit including:

the introduced cell according to <4> above.

The present disclosure can solve the above problems in the art to achieve the above object, and can provide a cell that stably expresses replicon RNA of a coronavirus and enables highly sensitive and highly reproducible screening for an anti-coronavirus therapeutic drug.

### BRIEF DESCRIPTION OF THE DRAWINGS

FIG. 1A is a view schematically illustrating a replicon-expressing vector as a whole;

FIG. 1B is a view illustrating details of a portion ranging from CMV to BGH in FIG. 1A;

FIG. 2A is a view illustrating results obtained through Western blotting analysis for expression of a replicon protein before and after transfection of a replicon-expressing vector and before and after addition of a drug;

FIG. 2B is a view illustrating results obtained through Northern blotting analysis for expression of replicon RNA before and after transfection of a replicon-expressing vector and before and after addition of a drug;

FIG. 2C is a view illustrating results obtained through reporter assay analysis for suppressive effects of drugs on replicon replication;

FIG. 3 is a view illustrating a producing method of a cell line of the present disclosure;

FIG. 4A is a view illustrating results obtained through reporter assay analysis for replicon expression in Huh7 cells and clone cells derived from Huh7.5.1 cells;

FIG. 4B is a view illustrating results obtained through RT-PCR analysis for expression of mRNA of a replicon protein in clone cells derived from Huh7.5.1 cells;

FIG. 4C is a view illustrating results obtained through reporter assay analysis for replicon expression in clone cells derived from VeroE6 cells;

FIG. 4D is a view illustrating results obtained through RT-PCR analysis for expression of mRNA of a replicon protein in clone cells derived from VeroE6 cells;

FIG. 5A is a view illustrating results obtained through Western blotting analysis for expression of a replicon protein before and after addition of a drug to cell line Huh7.5.1/Rep #4;

FIG. 5B is a view illustrating results obtained through Western blotting analysis for expression of a replicon protein before and after addition of a drug to cell line VeroE6/Rep #3;

FIG. 5C is a view illustrating results obtained through Northern blotting analysis for expression of replicon RNA in cell line VeroE6/Rep #3;

FIG. 6A is a view illustrating results obtained through crystal violet staining of cell line VeroE6/Rep #3;

FIG. 6B is a view illustrating results obtained through reporter assay analysis for replicon expression in cell line VeroE6/Rep #3;

FIG. 7A is a view illustrating results obtained through Northern blotting analysis for expression of replicon RNA before and after addition of a drug to cell line VeroE6/Rep #3;

FIG. 7B is a view illustrating results obtained through reporter assay analysis for replicon expression before and after addition of a drug to cell line VeroE6/Rep #3;

FIG. 8A is a view illustrating results obtained through screening using compound library 1;

FIG. 8B is an enlarged view about 75 compounds and EIDD-2801 in FIG. 8A;

FIG. 8C is a view illustrating results obtained through screening using compound library 2; and

FIG. 8D is an enlarged view about 41 compounds and EIDD-2801 in FIG. 8C.

### DESCRIPTION OF THE EMBODIMENTS

### (Cell Line)

The cell line is a cell line of Accession No. NITE P-03458.

The cell line of Accession No. NITE P-03458 is a cell line that stably expresses replicon RNA of a coronavirus.

The cell line of Accession No. NITE P-03458 can be produced by the following producing method of the cell line.

<Cell Line of Accession No. NITE P-03458>
1) Name of depositary institution: National Institute of Technology and Evaluation, Patent Microorganisms Depositary, Biological Resource Center, (postal code: 292-0818), 2-5-8 Kazusakamatari, Kisarazu-shi, Chiba, Japan
2) Receipt date: Apr. 8, 2021

### 3) Receipt No.: NITE AP-03458

### 4) Accession No.: NITE P-03458

### (Producing Method of the Cell Line)

The producing method of the cell line includes a transfection step and a selection step, and may further include other steps.

### <Transfection Step>

The transfection step is a step of transfecting a vector into a cell, where the vector includes a nucleic acid sequence encoding a nonstructural protein of a coronavirus, a reporter protein including a drug-resistant protein, and a structural protein of the coronavirus.

### <<Coronavirus>>

The coronavirus is not particularly limited and may be appropriately selected depending on the intended purpose. Examples thereof include viruses belonging to the family Coronaviridae, such as the subfamily Letovirinae and the subfamily Orthocoronavirinae.

Of these, viruses belonging to the subfamily Orthocoronavirinae are preferable.

The viruses belonging to the subfamily Orthocoronavirinae are not particularly limited and may be appropriately selected depending on the intended purpose. Examples thereof include viruses belonging to the genus Alphacoronavirus, the genus Betacoronavirus, the genus Deltacoronavirus, and the genus Gammacoronavirus.

Of these, viruses belonging to the genus Betacoronavirus are preferable.

The viruses belonging to the genus Betacoronavirus are not particularly limited and may be appropriately selected depending on the intended purpose. Examples thereof include viruses belonging to the subgenus Embecovirus, the subgenus Hibecovirus, the subgenus Merbecovirus, the subgenus Nobecovirus, and the subgenus Sarbecovirus.

Of these, viruses belonging to the subgenus Sarbecovirus are preferable.

The viruses belonging to the subgenus Sarbecovirus are not particularly limited and may be appropriately selected depending on the intended purpose. Examples thereof include SARS-related coronavirus (Severe acute respiratory syndrome-related coronavirus: SARSr-CoV), SARS coronavirus (Severe acute respiratory syndrome coronavirus: SARS-CoV), and the new coronavirus (Severe acute respiratory syndrome coronavirus 2: SARS-CoV-2 or COVID-19).

Of these, the new coronavirus (SARS-CoV-2 or COVID-19) is preferable.

### <<Vector>>

The vector includes a nucleic acid sequence encoding a nonstructural protein of the coronavirus, a reporter protein including a drug-resistant protein, and a structural protein of the coronavirus. The vector may further include other sequences.

The vector can be used as an expression vector of replicon RNA of the coronavirus.

In terms of easy handling, the vector preferably does not include all of the nonstructural protein, the structural protein, and accessary proteins of the coronavirus, and more preferably does not include all of the structural protein of the coronavirus.

The vector is not particularly limited and may be appropriately selected depending on the intended purpose. Examples thereof include a DNA vector and an RNA vector.

Of these, a DNA vector is preferable, and a cyclic DNA vector is more preferable.

The kind of the vector is not particularly limited and may be appropriately selected depending on the intended purpose. Examples thereof include an artificial chromosome, a plasmid, a cosmid, and a lambda phage.

Of these, an artificial chromosome is preferable, and a bacterial artificial chromosome (BAC) is more preferable.

### —Nonstructural Protein of the Coronavirus—

The nonstructural protein of the coronavirus is not particularly limited and may be appropriately selected depending on the intended purpose. Examples thereof include a polymerase, a replicase, and a protease, which are not responsible for formation of coronavirus particles and are produced by the coronavirus.

Of these, a polymerase or a replicase is preferable.

When the coronavirus is the new coronavirus (SARS-CoV-2 or COVID-19), the nonstructural protein of the coronavirus is not particularly limited and may be appropriately selected depending on the intended purpose. The nonstructural protein thereof is preferably ORF1a or ORF1ab, and ORF1ab is more preferable.

The nucleic acid sequence encoding the ORF1ab is not particularly limited and may be appropriately selected depending on the intended purpose. Examples thereof include a nucleic acid sequence having a sequence of SEQ ID NO. 1; i.e., a nucleic acid sequence having a sequence ranging from the 269^{th} residue to the 21558^{th} residue of SARS-CoV-2/Hu/DP/Kng/19-020 (GenBank ID: LC528232.1), a homolog of the nucleic acid sequence having the sequence of SEQ ID NO. 1, and a fragment of the nucleic acid sequence having a sequence of SEQ ID NO. 1.

The homolog of the nucleic acid sequence having the sequence of SEQ ID NO. 1 is not particularly limited and may be appropriately selected depending on the intended purpose. A nucleic acid sequence having 80% or higher sequence identity to the sequence of SEQ ID NO. 1 is preferable, a nucleic acid sequence having 85% or higher sequence identity thereto is more preferable, a nucleic acid sequence having 90% or higher sequence identity thereto is further more preferable, a nucleic acid sequence having 95% or higher sequence identity thereto is particularly preferable, and a nucleic acid sequence having 99% or higher sequence identity thereto is most preferable.

The fragment of the nucleic acid sequence having the sequence of SEQ ID NO. 1 is not particularly limited and may be appropriately selected depending on the intended purpose. Examples thereof include a partial sequence of the nucleic acid sequence having the sequence of SEQ ID NO. 1.

The partial sequence of the nucleic acid sequence having the sequence of SEQ ID NO. 1 is not particularly limited and may be appropriately selected depending on the intended purpose. A nucleic acid sequence having 50% or higher sequence identity to the sequence of SEQ ID NO. 1 is preferable, a nucleic acid sequence having 60% or higher sequence identity thereto is more preferable, a nucleic acid sequence having 65% or higher sequence identity thereto is further more preferable, a nucleic acid sequence having 70% or higher sequence identity thereto is particularly preferable, and a nucleic acid sequence having 75% or higher sequence identity thereto is most preferable.

### —Reporter Protein Including the Drug-Resistant Protein—

The drug-resistant protein is not particularly limited and may be appropriately selected depending on the intended purpose. Examples thereof include a G418-resistant protein, a puromycin-resistant protein, a blasticidin-resistant protein, a hygromycin B-resistant protein, a phleomycin-resistant protein, and a zeocin-resistant protein.

Of these, a G418-resistant protein is preferable.

In addition to the drug-resistant protein, the reporter protein including the drug-resistant protein preferably includes a luminescent protein or a fluorescent protein and more preferably includes a luminescent protein.

The reporter protein including the drug-resistant protein may be a fusion protein of the drug-resistant protein and the luminescent protein or the fluorescent protein, and is more preferably a fusion protein of the drug-resistant protein and the luminescent protein.

The luminescent protein is not particularly limited and may be appropriately selected depending on the intended purpose. Examples thereof include luminescent enzymes such as Renilla luciferase and firefly luciferase.

The fluorescent protein is not particularly limited and may be appropriately selected depending on the intended purpose. Examples thereof include a green fluorescent protein, a yellow fluorescent protein, a red fluorescent protein, and a blue fluorescent protein.

A nucleic acid sequence encoding the fusion protein of the drug-resistant protein and the luminescent protein is not particularly limited and may be appropriately selected depending on the intended purpose. Examples thereof include a nucleic acid sequence having a sequence of SEQ ID NO. 2 (Renilla luciferase-neomycin resistant gene: Reo gene), a homolog of the nucleic acid sequence having the sequence of SEQ ID NO. 2, and a fragment of the nucleic acid sequence having the sequence of SEQ ID NO. 2.

The homolog of the nucleic acid sequence having the sequence of SEQ ID NO. 2 is not particularly limited and may be appropriately selected depending on the intended purpose. A nucleic acid sequence having 80% or higher sequence identity to the sequence of SEQ ID NO. 2 is preferable, a nucleic acid sequence having 85% or higher sequence identity thereto is more preferable, a nucleic acid sequence having 90% or higher sequence identity thereto is further more preferable, a nucleic acid sequence having 95% or higher sequence identity thereto is particularly preferable, and a nucleic acid sequence having 99% or higher sequence identity thereto is most preferable.

The fragment of the nucleic acid sequence having the sequence of SEQ ID NO. 2 is not particularly limited and may be appropriately selected depending on the intended purpose. Examples thereof include a partial sequence of the nucleic acid sequence having the sequence of SEQ ID NO. 2.

The partial sequence of the nucleic acid sequence having the sequence of SEQ ID NO. 2 is not particularly limited and may be appropriately selected depending on the intended purpose. A nucleic acid sequence having 50% or higher sequence identity to the sequence of SEQ ID NO. 2 is preferable, a nucleic acid sequence having 60% or higher sequence identity thereto is more preferable, a nucleic acid sequence having 65% or higher sequence identity thereto is further more preferable, a nucleic acid sequence having 70% or higher sequence identity thereto is particularly preferable, and a nucleic acid sequence having 75% or higher sequence identity thereto is most preferable.

### —Structural Protein of the Coronavirus—

The structural protein of the coronavirus is not particularly limited and may be appropriately selected depending on the intended purpose. Examples thereof include a capsid protein, an envelope protein, a spike protein, and a membrane protein, which are responsible for formation of coronavirus particles and are produced by the coronavirus.

Of these, a capsid protein is preferable.

In terms of easy handling, the vector preferably does not include all of the structural protein of the coronavirus. In other words, the vector preferably does not include any one of the envelope protein, the spike protein, and the membrane protein, and the vector more preferably does not include the envelope protein, the spike protein, and the membrane protein.

When the coronavirus is the new coronavirus (SARS-CoV-2 or COVID-19), the structural protein of the coronavirus is not particularly limited and may be appropriately selected depending on the intended purpose. The structural protein thereof is preferably an N protein, an E protein, an S protein, or an M protein, and an N protein is more preferable.

In terms of easy handling, the vector preferably does not include all of the structural protein of the coronavirus. In other words, the vector preferably does not include any one of the E protein, the S protein, and the M protein, and the vector more preferably does not include the E protein, the S protein, and the M protein.

The nucleic acid sequence encoding the N protein is not particularly limited and may be appropriately selected depending on the intended purpose. Examples thereof include a nucleic acid sequence having a sequence of SEQ ID NO. 3 (a gene encoding the N protein: N gene), a homolog of the nucleic acid sequence having the sequence of SEQ ID NO. 3, and a fragment of the nucleic acid sequence having the sequence of SEQ ID NO. 3.

The homolog of the nucleic acid sequence having the sequence of SEQ ID NO. 3 is not particularly limited and may be appropriately selected depending on the intended purpose. A nucleic acid sequence having 80% or higher sequence identity to the sequence of SEQ ID NO. 3 is preferable, a nucleic acid sequence having 85% or higher sequence identity thereto is more preferable, a nucleic acid sequence having 90% or higher sequence identity thereto is further more preferable, a nucleic acid sequence having 95% or higher sequence identity thereto is particularly preferable, and a nucleic acid sequence having 99% or higher sequence identity thereto is most preferable.

The fragment of the nucleic acid sequence having the sequence of SEQ ID NO. 3 is not particularly limited and may be appropriately selected depending on the intended purpose. Examples thereof include a partial sequence of the nucleic acid sequence having the sequence of SEQ ID NO. 3.

The partial sequence of the nucleic acid sequence having the sequence of SEQ ID NO. 3 is not particularly limited and may be appropriately selected depending on the intended purpose. A nucleic acid sequence having 50% or higher sequence identity to the sequence of SEQ ID NO. 3 is preferable, a nucleic acid sequence having 60% or higher sequence identity thereto is more preferable, a nucleic acid sequence having 65% or higher sequence identity thereto is further more preferable, a nucleic acid sequence having 70% or higher sequence identity thereto is particularly preferable, and a nucleic acid sequence having 75% or higher sequence identity thereto is most preferable.

### —Other Sequences—

The other sequences are not particularly limited and may be appropriately selected depending on the intended purpose. Examples thereof include a promoter sequence, 5′-UTR, 3′-UTR, a poly(A) sequence, a Ribozyme sequence, a transcription termination signal, a regulatory sequence, a restriction enzyme sequence, and a spacer sequence.

The regulatory sequence is not particularly limited and may be appropriately selected depending on the intended purpose. The regulatory sequence preferably includes a sequence of ACGAAC (SEQ ID NO. 4), more preferably includes a sequence of ACGAAC (SEQ ID NO. 4) and a sequence of the latter 129 bp (SEQ ID NO. 5) of an ORF8 gene, and further preferably includes a sequence of ACGAAC (SEQ ID NO. 4), a sequence of the latter 129 bp (SEQ ID NO. 5) of an ORF8 gene, and a sequence of AAACTAAA (SEQ ID NO. 6).

### <<Cell>>

The cell is not particularly limited and may be appropriately selected depending on the intended purpose. Examples thereof include an animal cell, an insect cell, a plant cell, and a yeast cell.

Of these, an animal cell is preferable.

The animal cell is not particularly limited and may be appropriately selected depending on the intended purpose. Examples thereof include a VeroE6 cell, a Huh7 cell, a Huh7.5.1 cell, an HEK293T cell, an HEK293 cell, a Hela cells, a CHO cells, an iPS cell, and a mesenchymal stem cell.

Of these, a VeroE6 cell, a Huh7 cell, or a Huh7.5.1 cell is preferable, a VeroE6 cell or a Huh7.5.1 cell is more preferable, and a VeroE6 cell is further preferable.

The animal cell to be used may be a commercially available product such as HEK293T (ATCC #CRL-11268) or VeroE6 (ATCC #CRL-1586).

### <<Transfection>>

A method for the transfection is not particularly limited and may be appropriately selected depending on the intended purpose. Examples thereof include lipofection, calcium phosphate transfection, electroporation, and microinjection.

Of these, lipofection is preferable.

### <Selection Step>

The selection step is a step of performing selection on the cell obtained in the transfection step using a drug.

A method for the selection is not particularly limited and may be appropriately selected depending on the intended purpose. Examples thereof include a method of adding a drug to the culture medium of the cell obtained in the transfection step, followed by culturing the cell.

When the nucleic acid sequence included in the vector and encoding the reporter protein including the drug-resistant protein is a nucleic acid sequence encoding the reporter protein including the G418-resistant protein, G418 can be added to the culture medium of the cell obtained in the transfection step.

The period of the selection is not particularly limited and may be appropriately selected depending on the intended purpose. The period thereof is preferably one week or longer, more preferably two weeks or longer, further preferably three weeks or longer, and particularly preferably four weeks or longer.

### <Other Steps>

The other steps are not particularly limited and may be appropriately selected depending on the intended purpose. Examples thereof include a culture step after the selection step, a cloning step after the selection step, and a cell-screening step after the selection step.

<<Culture Step after the Selection Step>>

The culture step after the selection step is not particularly limited and may be appropriately selected depending on the intended purpose. Examples thereof include a step of removing the drug from the culture medium in which the cell is cultured with the drug, followed by culturing the cell.

The period of the culture step after the selection step is not particularly limited and may be appropriately selected depending on the intended purpose. The period thereof is preferably one week or longer and more preferably two weeks or longer.

<<Cloning Step after the Selection Step>>

The cloning step after the selection step is not particularly limited and may be appropriately selected depending on the intended purpose. Examples thereof include a step of cloning cells derived from a single cell through limiting dilution.

<<Cell-Screening Step after the Selection Step>>

The cell-screening step after the selection step is not particularly limited and may be appropriately selected depending on the intended purpose. Examples thereof include a step of screening the cell using, as an index, the expression level of the nonstructural protein of the coronavirus or the structural protein of the coronavirus or the expression level of the nucleic acid sequence encoding the nonstructural protein of the coronavirus or the nucleic acid sequence encoding the structural protein of the coronavirus.

When the reporter protein includes the luminescent protein or the fluorescent protein, the cell can be screened using, as an index, luminescence or fluorescence therefrom.

### (Introduced Cell)

The introduced cell is a cell into which the vector is introduced.

The vector is as described above.

The introduced cell is not particularly limited and may be appropriately selected depending on the intended purpose. The introduced cell is preferably a cell that expresses the replicon RNA of the coronavirus and more preferably a cell that expresses the replicon RNA of the coronavirus and the structural protein of the coronavirus.

The introduced cell can be produced by the producing method of the cell line.

One example of the introduced cell is the cell line of Accession No. NITE P-03458, which expresses the replicon RNA of the coronavirus and the structural protein of the coronavirus.

### (Screening Method for the Anti-Coronavirus Therapeutic Drug)

The screening method for the anti-coronavirus therapeutic drug is a screening method using the introduced cell.

The introduced cell is as described above.

The screening method for the anti-coronavirus therapeutic drug is not particularly limited and may be appropriately selected depending on the intended purpose. Examples thereof include a method of adding a candidate of the anti-coronavirus therapeutic drug to a culture medium of the introduced cell and screening the anti-coronavirus therapeutic drug using, as an index, the expression level of the nonstructural protein of the coronavirus of the introduced cell or the structural protein of the coronavirus of the introduced cell or the expression level of the nucleic acid sequence encoding the nonstructural protein of the coronavirus of the introduced cell or the nucleic acid sequence encoding the structural protein of the coronavirus of the introduced cell.

When the reporter protein includes the luminescent protein or the fluorescent protein, the anti-coronavirus therapeutic drug can be screened using, as an index, luminescence or fluorescence therefrom.

In the screening using, as an index, the expression level of the protein or the nucleic acid, the luminescence or fluorescence, etc., the effectiveness rate of a compound can be calculated through standardization on the basis of the cell survival rate of the introduced cell.

### (Anti-Coronavirus Therapeutic Drug Screening Kit)

The anti-coronavirus therapeutic drug screening kit includes the introduced cell and can further include other components.

The introduced cell is as described above.

The other components are not particularly limited and may be appropriately selected depending on the intended purpose. Examples thereof include a protocol, a written instruction, a culture medium, and an additive to the medium.

### EXAMPLES

The present disclosure will be described by way of Examples, but the present disclosure should not be construed as being limited to these Examples.

### Example 1 Production of Replicon RNA-Expressing Vector pBAC-SCoV2-Rep-Reo

The above expression vector of the full length was divided into the following 10 fragments (fragment 1 to fragment 10). The fragments were each amplified through PCR and ligated through Circular Polymerase Extension Reaction (Journal of Virology, 2013 87:2367-2372). The obtained ligates were introduced into Escherichia coli competent cells (BAC-Optimized Replicator v2.0 Electrocompetent Cells: obtained from Lucigen Corporation) followed by cloning.

The fragment 1 (SEQ ID NO. 7) includes a part of the ORF1ab gene (the sequence ranging from the 389^{th} residue to the 1661^{st} residue of SEQ ID NO. 1); i.e., the sequence ranging from the 657^{th} residue to the 1929^{th} residue of SARS-CoV-2 (GenBank ID: LC528232.1).

The fragment 2 (SEQ ID NO. 8) includes a part of the ORF1ab gene (the sequence ranging from the 1632^{nd} residue to the 4054^{th} residue of SEQ ID NO. 1); i.e., the sequence ranging from the 1900^{th} residue to 4322^{nd} residue of SARS-CoV-2 (GenBank ID: LC528232.1).

The fragment 3 (SEQ ID NO. 9) includes a part of the ORF1ab gene (the sequence ranging from the 4025^{th} residue to the 6494^{th} residue of SEQ ID NO. 1); i.e., the sequence ranging from the 4293^{th} residue to the 6762^{nd} residue of SARS-CoV-2 (GenBank ID: LC528232.1).

The fragment 4 (SEQ ID NO. 10) includes a part of the ORF1ab gene (the sequence ranging from the 6465^{th} residue to the 8595^{th} residue of SEQ ID NO. 1); i.e., the sequence ranging from the 6733^{rd} residue to the 8863^{rd} residue of SARS-CoV-2 (GenBank ID: LC528232.1).

The fragment 5 (SEQ ID NO. 11) includes a part of the ORF1ab (the sequence ranging from the 8566^{th} residue to the 10829^{th} residue of SEQ ID NO. 1); i.e., the sequence ranging from the 8834^{th} residue to the 11097^{th} residue of SARS-CoV-2 (GenBank ID: LC528232.1).

The fragment 6 (SEQ ID NO. 12) includes a part of the ORF1ab gene (the sequence ranging from the 10800^{th} residue to the 13601^{st} residue of SEQ ID NO. 1); i.e., the sequence ranging from the 11068^{th} residue to the 13869^{th} residue of SARS-CoV-2 (GenBank ID: LC528232.1).

The fragment 7 (SEQ ID NO. 13) includes a part of the ORF1ab gene (the sequence ranging from the 13569^{th} residue to the 17084^{th} residue of SEQ ID NO. 1); i.e., the sequence ranging from the 13837^{th} residue to the 17352^{nd} residue of SARS-CoV-2 (GenBank ID: LC528232.1).

The fragment 8 (SEQ ID NO. 14) includes a part of the ORF1ab gene (the sequence ranging from the 17053^{rd} residue to 21290^{th} residue of SEQ ID NO. 1); i.e., the sequence ranging from the 17307^{th} residue to 21558^{th} residue of SARS-CoV-2 (GenBank ID: LC528232.1), and an artificial spacer sequence.

The fragment 9 (SEQ ID NO. 15) includes an artificial spacer sequence, a part of the ORF8 gene (SEQ ID NO. 5); i.e., the sequence ranging from the 28134^{th} residue to 28262^{nd} residue of SARS-CoV-2 (GenBank ID: LC528232.1), the sequence of SEQ ID NO. 4, the sequence of SEQ ID NO. 6, and the sequence of the Reo gene (SEQ ID NO. 2).

The fragment 10 (SEQ ID NO. 16) includes a part of the ORF8 gene (SEQ ID NO. 5); i.e., the sequence ranging from the 28134^{th} residue to 28262^{nd} residue of SARS-CoV-2 (GenBank ID: LC528232.1), the sequence of SEQ ID NO. 4, the sequence of SEQ ID NO. 6, the sequence ranging from the N gene (SEQ ID NO. 3) to 3′UTR; i.e., the sequence ranging from the 28277^{th} residue to 29873^{rd} residue of SARS-CoV-2 (GenBank ID: LC528232.1), the sequence ranging from the poly(A) sequence to the transcription termination signal (BGH), the full-length sequence of pSMART BAC 2.0 vector, the CMV promoter sequence, and the sequence ranging from 5′UTR to a part of the ORF1ab gene (the sequence ranging from the 1^{st} residue to the 418^{th} residue of SEQ ID NO. 1); i.e., the sequence ranging from the 1^{st} residue to the 686^{th} residue of SARS-CoV-2 (GenBank ID: LC528232.1).

The fragment 1 was amplified through PCR using SARS-CoV-2 genome cDNA as a template, and primer 1 (SEQ ID NO. 17 as a forward primer) and primer 2 (SEQ ID NO. 18 as a reverse primer).

The fragment 2 was amplified through PCR using SARS-CoV-2 genome cDNA as a template, and primer 3 (SEQ ID NO. 19 as a forward primer) and primer 4 (SEQ ID NO. 20 as a reverse primer).

The fragment 3 was amplified through PCR using SARS-CoV-2 genome cDNA as a template, and primer 5 (SEQ ID NO. 21 as a forward primer) and primer 6 (SEQ ID NO. 22 as a reverse primer).

The fragment 4 was amplified through PCR using SARS-CoV-2 genome cDNA as a template, and primer 7 (SEQ ID NO. 23 as a forward primer) and primer 8 (SEQ ID NO. 24 as a reverse primer).

The fragment 5 was amplified through PCR using SARS-CoV-2 genome cDNA as a template, and primer 9 (SEQ ID NO. 25 as a forward primer) and primer 10 (SEQ ID NO. 26 as a reverse primer).

The fragment 6 was amplified through PCR using SARS-CoV-2 genome cDNA as a template, and primer 11 (SEQ ID NO. 27 as a forward primer) and primer 12 (SEQ ID NO. 28 as a reverse primer).

The fragment 7 was amplified through PCR using SARS-CoV-2 genome cDNA as a template, and primer 13 (SEQ ID NO. 29 as a forward primer) and primer 14 (SEQ ID NO. 30 as a reverse primer).

The fragment 8 was amplified through PCR using SARS-CoV-2 genome cDNA as a template, and primer 15 (SEQ ID NO. 31 as a forward primer) and primer 16 (SEQ ID NO. 32 as a reverse primer).

The fragment 9 was amplified through PCR using a Reo gene subcloning vector as a template, and primer 17 (SEQ ID NO. 33 as a forward primer) and primer 18 (SEQ ID NO. 34 as a reverse primer).

The fragment 10 was amplified through PCR using an artificial gene as a template, and primer 19 (SEQ ID NO. 35 as a forward primer) and primer 20 (SEQ ID NO. 36 as a reverse primer), the artificial gene including pSMART BAC 2.0, SARS-CoV-2 genome cDNA, a poly(A) sequence (pA), a Ribozyme sequence (Rz), and a transcription termination signal (BGH).

The SARS-CoV-2 genome cDNA was kindly provided by Professor Toru Okamoto at Institute for Advanced Co-Creation Studies, Osaka University.

The Reo gene subcloning vector was synthesized through PCR using a pGL4.72 reporter vector (manufactured by Promega Corporation, #E6901) and a pcDNA3.1 cloning vector (manufactured by Invitrogen Inc., #V79020).

The pSMART BAC 2.0 was purchased from Lucigen Corporation (BAC-Optimized Replicator v2.0).

The poly(A) sequence (pA) was synthesized using oligo DNA synthesis service (provided by Fasmac).

The Ribozyme sequence (Rz) was synthesized using oligo DNA synthesis service (provided by Fasmac).

As the artificial gene including the transcription termination signal (BGH), a pcDNA3.1 cloning vector (manufactured by Invitrogen Inc., #V79020) was used.

The PCR was performed with 30 cycles at an annealing temperature of 60° C. using PrimeSTAR GXL DNA Polymerase (manufactured by Takara Bio Inc., #R050).

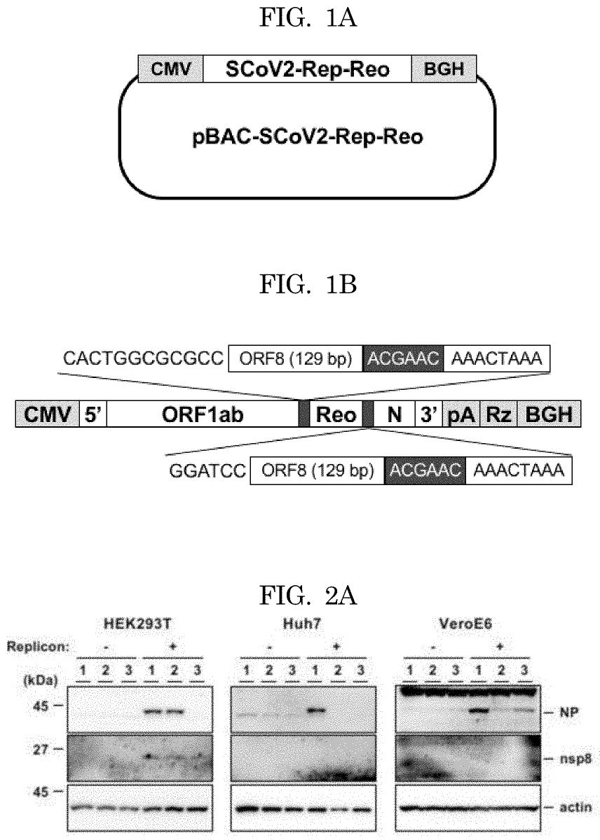

In the above-described manner, a construct illustrated in FIG. 1A (pBAC-SCoV2-Rep-Reo) was produced (SEQ ID NO. 37).

As illustrated in FIG. 1A, the pBAC-SCoV2-Rep-Reo is a DNA cloning vector (pSMART BAC 2.0; black line) into which the promoter (CMV), the new coronavirus replicon (SCoV-Rep-Reo), and the transcription termination signal (BGH) are inserted.

FIG. 1B illustrates details of a portion ranging from CMV to BGH in FIG. 1A.

The 5′-UTR (5′), the ORF1ab, the N protein (N), and the 3′-UTR (3′) are regions necessary for replication of the genome derived from the new coronavirus (COVID-19).

In FIG. 1B, the “ORF8 (129 bp) ACGAAC AAACTAAA” upstream of the Reo gene is a nucleic acid sequence derived from the SARS-CoV2 genome and is a regulatory sequence necessary for expressing the Reo gene.

The ORF8 (129 bp) is the latter 129 bp of the ORF8 gene, and the ACGAAC is a transcription regulation sequence (TRS). The “CACTGGCGCGCC” is an artificial spacer sequence including a restriction enzyme sequence for confirmation.

In FIG. 1B, the “ORF8 (129 bp) ACGAAC AAACTAAA” downstream of the Reo gene is a nucleic acid sequence derived from the SARS-CoV2 genome and is a regulatory sequence necessary for expressing the N gene.

The ORF8 (129 bp) is the latter 129 bp of the ORF8 gene, and the ACGAAC is a transcription regulation sequence (TRS). The “GGATCC” is a restriction enzyme sequence inserted for confirmation.

### Example 2 Introduction of Replicon RNA-Expressing Vector pBAC-SCoV2-Rep-Reo

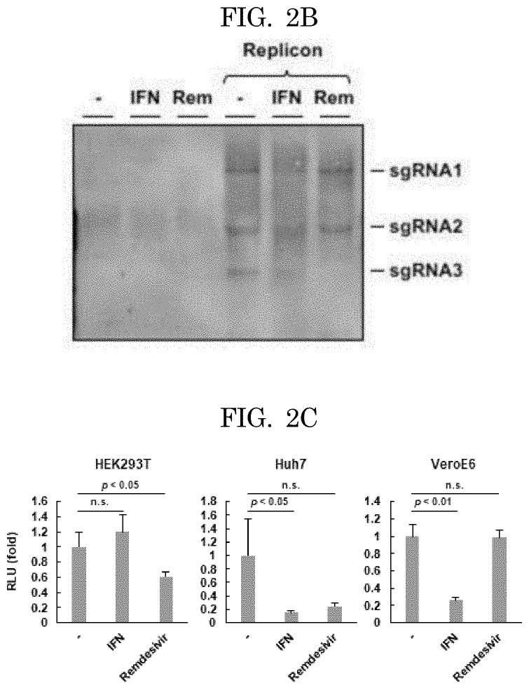

The pBAC-SCoV2-Rep-Reo was transfected into culture cells (HEK293T cells, Huh7 cells, or VeroE6 cells) through lipofection as described below. A cell lysate was prepared, and the expression levels of the N protein, nsp8, and actin were analyzed through Western blotting (FIG. 2A).

An anti-N protein antibody used was kindly provided by Professor Toru Okamoto at Institute for Advanced Co-Creation Studies, Osaka University. An anti-nsp8 antibody used was Anti-SARS-CoV/SARS-CoV-2 (COVID-19) NSP8 (#GTX632696) (manufactured by GeneTex Inc.). An anti-actin antibody used was Monoclonal Anti-β-Actin antibody (#A5441) (Sigma-Aldrich Co.).

In FIG. 2A, lane 1 is of a cell sample to which no drug was added, lane 2 is of a cell sample to which interferon was added, and lane 3 is of a cell sample to which remdesivir was added.

Also, the pBAC-SCoV2-Rep-Reo was transfected into the HEK293T cells through lipofection as described below. RNA was prepared, and the expression levels of subgenomic RNA 1 (pp1a, pp1ab), subgenomic RNA 2 (reporter gene), and subgenomic RNA 3 (N gene) were analyzed through Northern blotting (FIG. 2B).

The subgenomic RNAs 1 to 3 were detected using a common RNA probe complementary to the N gene.

The RNA probe was synthesized using the DNA sequence of SEQ ID NO. 38 as a template in RNA probe synthesis.

In FIG. 2B, the “-” indicates the cell sample to which no drug was added, the “IFN” indicates the cell sample to which interferon was added, and the “Rem” indicates the cell sample to which remdesivir was added.

Moreover, the pBAC-SCoV2-Rep-Reo was transfected into the culture cells (HEK293T cells, Huh7 cells, or VeroE6 cells) through lipofection as described below, and the suppressive effect on replicon replication was analyzed through reporter assay (FIG. 2C).

A statistically significant difference was evaluated based on the Student's t-test. The “n.s” indicates that there is no statistically significant difference.

As illustrated in FIG. 2C, the activity of Renilla luciferase was confirmed to be reduced by administration of interferon or remdesivir, which are reported to inhibit the replication of the new coronavirus (COVID-19). This confirmed that the intracellular replicon RNA replication level could be monitored through simple reporter assay (Renilla luciferase assay) by the pBAC-SCoV2-Rep-Reo.

### —Transfection—

The HEK293T and Huh7 cells were kindly provided by Professor Yoshiharu Matsuura at Research Institute for Microbial Diseases, Osaka University. The VeroE6 cells were obtained from American Type Culture Collection (ATCC #CRL-1586).

The culture cells were seeded to a multi-well plate such as a 24-well plate or a 6-well plate, and cultured at 37° C. overnight in the presence of 5% CO₂.

About 16 hours after, the pBAC-SCoV2-Rep-Reo and TransIT-LT1 (Mirus Bio LLC) in an amount three times that of DNA were mixed, and the mixture was incubated at room temperature for 30 minutes.

The mixture was added to the culture cells after the medium had been changed, and the culture cells were cultured at 37° C. for one or two days in the presence of 5% CO₂.

### —Preparation of Cell Lysate—

One or two days after the transfection, the culture cells in the culture plate were washed with PBS. A cell lysis solution (50 mM Tris-HCl (pH 7.5), 150 mM NaCl, 1% Nonidet-P40, 0.5% sodium deoxycholate, 0.1% SDS, complete protease inhibitor cocktail (Roche Co.)) was added. The cells into which the replicon RNA-expressing vector had been transiently introduced and the cells which had not been transfected were scraped with a scraper. The cells were treated on ice for 20 minutes, followed by centrifuging to recover a supernatant, to obtain a cell lysate.

Six hours after the transfection, the medium was changed. The cells which had been exposed to interferon (100 U/mL, manufactured by PeproTech, Inc.) or remdesivir (5 μg/mL, manufactured by Cayman Chemical Company) for one or two days, the cells which had not been transfected, and the cells which had not been transfected and had been exposed to interferon (100 U/mL, manufactured by PeproTech, Inc.) or remdesivir (5 μg/mL, manufactured by Cayman Chemical Company) for two days were treated in the same manner to obtain cell lysates.

### —Preparation of RNA—

One or two days after the transfection, the culture cells in the culture plate were washed with PBS, followed by addition of 1 mL of TRIzol (manufactured by Invitrogen Inc.). The cells to which the replicon RNA-expressing vector had been transiently introduced were lysed to purify and obtain RNA.

Six hours after the transfection, the cells which had been exposed to interferon (100 U/mL, manufactured by PeproTech, Inc.) or remdesivir (5 μg/mL, manufactured by Cayman Chemical Company) for one or two days, the cells which had not been transfected, and the cells which had not been transfected and had been exposed to interferon (100 U/mL, manufactured by PeproTech, Inc.) or remdesivir (5 μg/mL, manufactured by Cayman Chemical Company) for two days were treated in the same manner to obtain RNA.

### —Reporter Assay—

One or two days after the transfection, the culture cells in the culture dish were washed with PBS, followed by addition of Renilla Luciferase Assay Lysis Buffer (manufactured by Promega Corporation) which had been diluted. The culture cells were scraped with a scraper and treated at room temperature for 15 minutes, followed by centrifuging to recover a supernatant, to obtain a cell lysate.

The luciferase activity of the cell lysate was measured using Renilla Luciferase Assay System (manufactured by Promega Corporation, #E2810).

Six hours after the transfection, the cells which had been exposed to interferon (100 U/mL, manufactured by PeproTech, Inc.) or remdesivir (5 μg/mL, manufactured by Cayman Chemical Company) for one or two days, the cells which had not been transfected, and the cells which had not been transfected and had been exposed to interferon (100 U/mL, manufactured by PeproTech, Inc.) or remdesivir (5 μg/mL, manufactured by Cayman Chemical Company) for two days were subjected to the reporter assay in the same manner.

### Example 3 Production of Replicon RNA-Expressing Cell Line

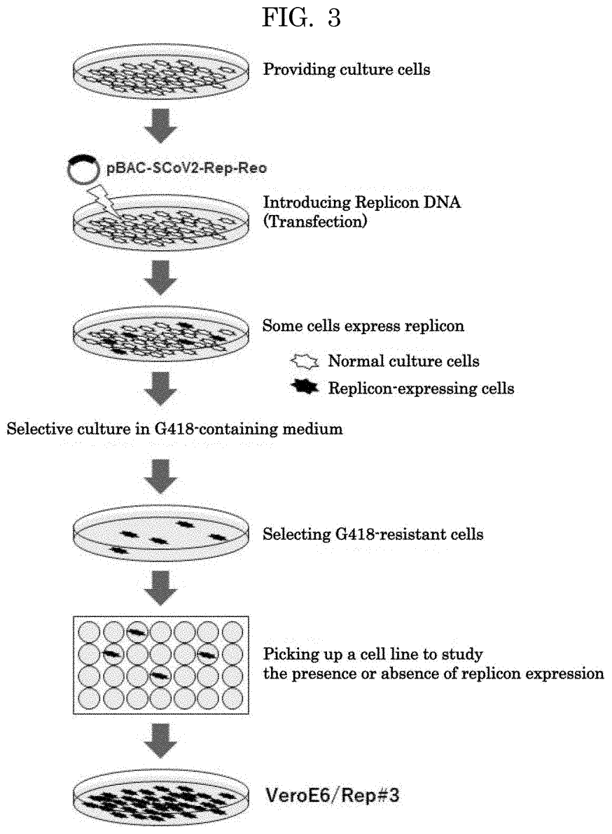

In accordance with the steps illustrated in FIG. 3, a replicon RNA-expressing cell line was produced.

In the same manner as in Example 2, the pBAC-SCoV2-Rep-Reo was transfected into the culture cells (Huh7 cells, Huh7.5.1 cells, or VeroE6 cells) through lipofection. Then, the culture cells were cultured in a culture medium containing G418 (neomycin (#09380), manufactured by NACALAI TESQUE, INC.) (1 mg/mL).

The Huh7.5.1 cells were kindly provided by Professor Ralf Bartenschlager at Heidelberg University.

The G418 kills the culture cells lacking expression of G418-resistant gene. The culture cells expressing the reporter gene (Reo gene) derived from the pBAC-SCoV2-Rep-Reo acquire resistance to G418, and are able to grow in the G418-containing culture medium.

After the culture in the G418-containing culture medium, cell colonies resistant to G418 were picked up, and cell lines (clone cells) were obtained.

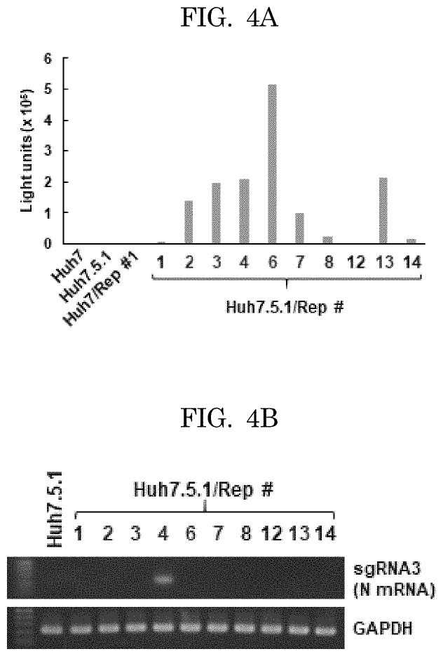

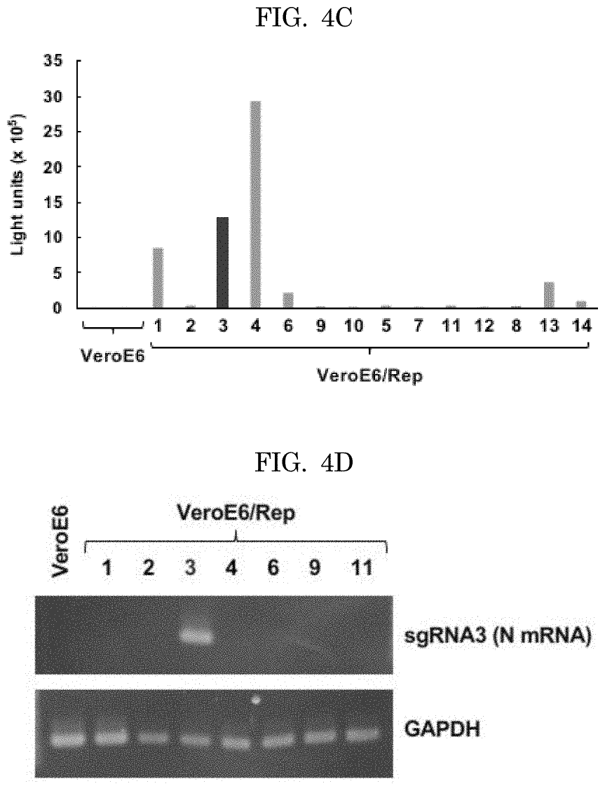

FIG. 4A indicates the results of the reporter assay (Renilla luciferase assay) in the cell line derived from the Huh7 (clone #1) and the cell lines derived from the Huh7.5.1 cells (clone #1 to 4, 6 to 8, and 12 to 14). The reporter assay was performed in the same manner as in Example 2.

Some of the Huh7.5.1/Rep cell lines including clone #4 derived from the Huh7.5.1 cells (Huh7.5.1/Rep #4 cells) exhibited high luciferase activity.

FIG. 4B indicates the analysis results obtained through RT-PCR for the replicon RNA (N protein) in the cell lines derived from the Huh7.5.1 cells (clone #1 to 4, 6 to 8, and 12 to 14), and GAPDH of the host cells as a control. Preparation of RNA was performed with an RNeasy mini kit (manufactured by Qiagen).

Sustained expression of the replicon RNA could be confirmed in clone #4 derived from the Huh7.5.1 cells (Huh7.5.1/Rep #4 cells).

FIG. 4C indicates the results of the reporter assay (Renilla luciferase assay) in the cell lines derived from the VeroE6 cells (clone #1 to 14). The reporter assay was performed in the same manner as in Example 2.

Some of the VeroE6/Rep cell lines including clone #3 derived from the VeroE6 cells (VeroE6/Rep #3 cells: the cell line of Accession No. NITE P-03458) exhibited high luciferase activity.

FIG. 4D indicates the analysis results obtained through RT-PCR for the replicon RNA (N protein) in the cell lines derived from the VeroE6 cells (clone #1 to 4, 6, 9, and 11), and GAPDH of the host cells as a control. Preparation of RNA was performed with an RNeasy mini kit (manufactured by Qiagen).

Sustained expression of the replicon RNA could be confirmed in clone #3 derived from the VeroE6 cells (VeroE6/Rep #3 cells: the cell line of Accession No. NITE P-03458).

This confirmed that the Renilla luciferase and the replicon RNA were expressed in clone #4 derived from the Huh7.5.1 cells (Huh7.5.1/Rep #4 cells) and clone #3 derived from the VeroE6 cells (VeroE6/Rep #3 cells: the cell line of Accession No. NITE P-03458).

The RT-PCR of the mRNA encoding the N protein was performed using primer 21 (SEQ ID NO. 39 as a forward primer) and primer 22 (SEQ ID NO. 40 as a reverse primer).

The RT-PCR of the mRNA encoding the GAPDH was performed using primer 23 (SEQ ID NO. 41 as a forward primer) and primer 24 (SEQ ID NO. 42 as a reverse primer).

### Example 4 Expression of the Replicon Protein in the Cell Line

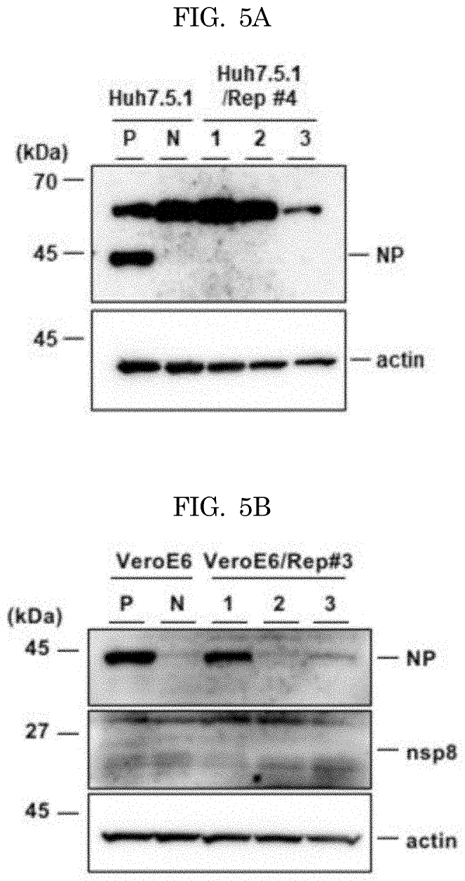

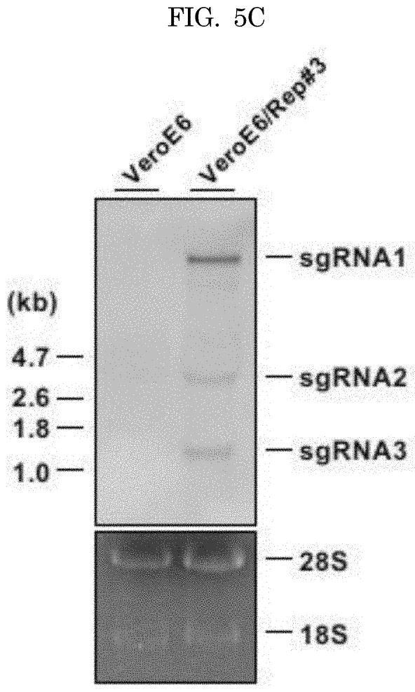

In the same manner as in Example 2, a cell lysate of the Huh7.5.1/Rep #4 cell line, and a cell lysate of the Huh7.5.1/Rep #4 cell line which had been exposed to interferon or remdesivir were prepared, and the expression levels of the N protein, nsp8, and actin were analyzed through Western blotting (FIG. 5A).

As a control of the N protein expression, the Huh7.5.1 cells transfected with the pBAC-SCoV2-Rep-Reo were analyzed in the same manner (FIG. 5A).

In FIG. 5A, lane P is of a sample the Huh7.5.1 cells transfected with the pBAC-SCoV2-Rep-Reo, lane N is of a sample of the Huh7.5.1 cells, lane 1 is of a sample of the Huh7.5.1/Rep #4 cell line, lane 2 is of a sample of the Huh7.5.1/Rep #4 cell line to which interferon was added, and lane 3 is of a sample of the Huh7.5.1/Rep #4 cell line to which remdesivir was added.

As illustrated in FIG. 5A, no expression of the N protein could be confirmed in the Huh7.5.1/Rep #4.

In the same manner as in Example 2, a cell lysate of the VeroE6/Rep #3 cell line, and a cell lysate of the VeroE6/Rep #3 cell line which had been exposed to interferon or remdesivir were prepared, and the expression levels of the N protein, nsp8, and actin were analyzed through Western blotting (FIG. 5B).

As a control of the N protein expression, the VeroE6 cells transfected with the pBAC-SCoV2-Rep-Reo were analyzed in the same manner (FIG. 5B).

In FIG. 5B, lane P is of a sample of the VeroE6 cells transfected with the pBAC-SCoV2-Rep-Reo, lane N is of a sample of the VeroE6 cells, lane 1 is of a sample of the VeroE6/Rep #3 cell line, lane 2 is of a sample of the VeroE6/Rep #3 cell line to which interferon was added, and lane 3 is of a sample of the VeroE6/Rep #3 cell line to which remdesivir was added.

As illustrated in FIG. 5B, the N protein was expressed in the VeroE6/Rep #3 cell line, and as a result of the addition of interferon or remdesivir, the expression level of the N protein was reduced.

In the same manner as in Example 2, RNA of the VeroE6 cells and the VeroE6/Rep #3 cell line were prepared, and the expression levels of the subgenomic RNA 1 (pp1a, pp1ab), the subgenomic RNA 2 (reporter gene), and the subgenomic RNA 3 (N gene) were analyzed through Northern blotting.

As a control of an amount of the RNA, 28S ribosomal RNA and 18S ribosomal RNA were analyzed through ethidium bromide staining (#E8751, manufactured by Sigma-Aldrich Co.) (FIG. 5C).

This suggests that the new coronavirus replicon is replicated in the VeroE6/Rep #3 cell line.

### Example 5 Stability of the VeroE6/Rep #3 Cell Line

Sustainability of the replicon expression of the VeroE6/Rep #3 cell line was confirmed.

The VeroE6 cells or cells of the VeroE6/Rep #3 cell line were cultured for two weeks in the absence of G418, then seeded to a 10 cm-dish, and cultured in a G418-containing culture medium for two weeks.

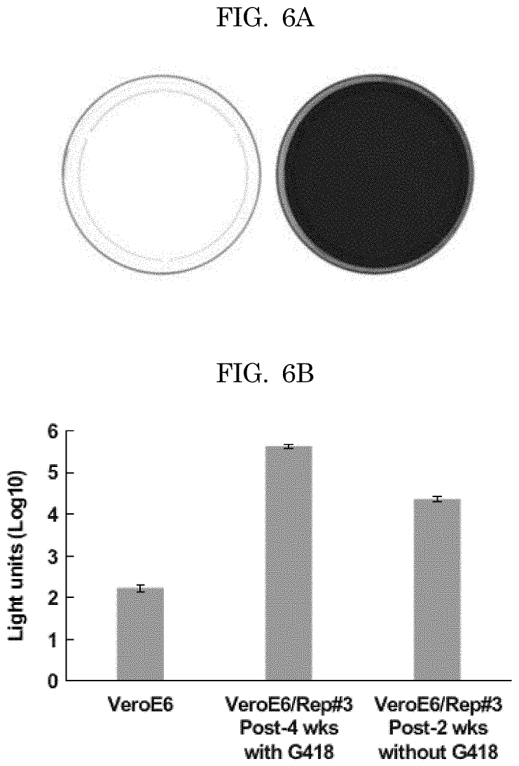

Surviving cells were stained in blue through crystal violet staining (031-04852, manufactured by FUJIFILM Wako Pure Chemical Corporation). The VeroE6 cells were killed (FIG. 6A, the left dish) while the VeroE6/Rep #3 exhibited resistance to the G418 (FIG. 6A, the right dish).

In the same manner as in Example 2, the Renilla luciferase assay was performed using the VeroE6 cells, the VeroE6/Rep #3 cells that had been cultured in the presence of G418 for four weeks or longer, and the VeroE6/Rep #3 cells that had been cultured in the absence of G418 for two weeks. High luciferase activity was maintained in the VeroE6/Rep #3 cells that had been cultured in the presence of G418 for four weeks or longer (FIG. 6B, middle).

Also in the VeroE6/Rep #3 cells that had been cultured in the absence of G418 for two weeks (FIG. 6B, right), higher activity was maintained than in the VeroE6 cells (FIG. 6B, left).

These results confirmed that the replicon expression of the VeroE6/Rep #3 stably continued for at least two weeks or longer in the absence of G418 and for at least four weeks or longer in the presence of G418.

### Example 6 Evaluation of Suitability of the VeroE6/Rep #3 Cell Line

Suitability of the VeroE6/Rep #3 cell line to drug screening was confirmed (the replication level of the new coronavirus replicon was monitored).

In the same manners as in Examples 2 and 4, RNA of the VeroE6/Rep #3 cell line and the VeroE6/Rep #3 cell line which had been exposed to interferon (100 U/mL, manufactured by PeproTech, Inc.) for three days or to remdesivir (5 μg/mL, manufactured by Cayman Chemical Company) for three days were prepared, and the expression levels of the subgenomic RNA 1 (pp1a, pp1ab), the subgenomic RNA 2 (reporter gene), and the subgenomic RNA 3 (N gene) were analyzed through Northern blotting.

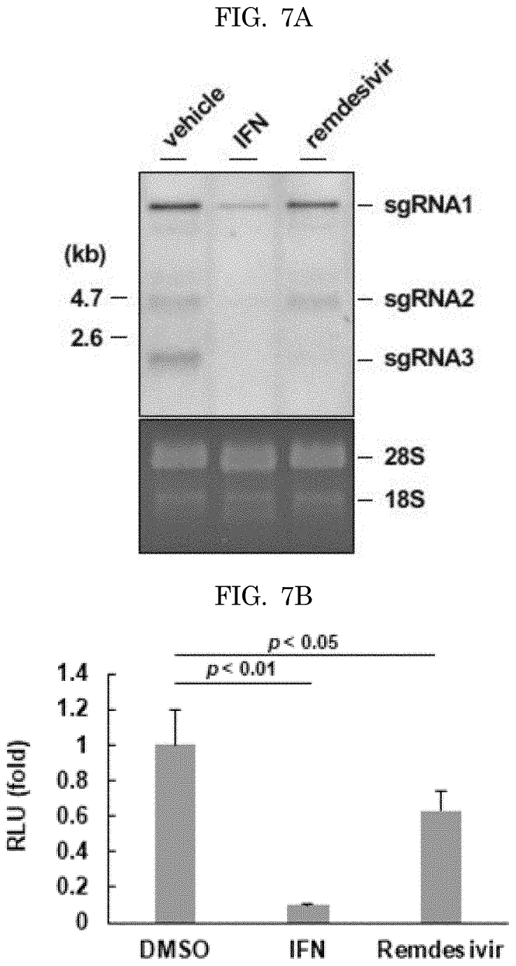

As a control of an amount of the RNA, 28S ribosomal RNA and 18S ribosomal RNA were analyzed through ethidium bromide staining (#E8751, manufactured by Sigma-Aldrich Co.) (FIG. 7A).

As illustrated in FIG. 7A, the intracellular replicon RNA was reduced when the VeroE6/Rep #3 cell line was treated with interferon or remdesivir, which are confirmed to inhibit the replication of the new coronavirus (COVID-19).

In the same manner as in Example 2, the reporter assay was performed using the VeroE6/Rep #3 cell line, and the VeroE6/Rep #3 cell line which had been exposed to interferon (100 U/mL, manufactured by PeproTech, Inc.) for three days or to remdesivir (5 μg/mL, manufactured by Cayman Chemical Company) for three days, in order to study whether the replication level could be monitored. The Renilla luciferase activity was reduced in the VeroE6/Rep #3 cell line to which interferon or remdesivir had been administered (FIG. 7B, middle and right).

A statistically significant difference was evaluated based on the Student's t-test.

Considerable reduction in the Renilla luciferase activity was observed in the administration of interferon, while mild reduction was observed in remdesivir. Such a difference in drug susceptibility was in agreement with the results of the VeroE6 cells transfected with the pBAC-SCoV2-Rep-Reo (FIGS. 2A and C).

Regarding the mild reduction in the Renilla luciferase activity by remdesivir, the drug susceptibility could be evaluated in the VeroE6/Rep #3 cell line with higher sensitivity than in the VeroE6 cells (FIG. 7B, p<0.05).

This suggests that a replicon system using the VeroE6/Rep #3 cell line has screening suitability comparable to the existing replicon system (in which a replicon-expressing vector is transiently transfected into cells) and can provide highly sensitive and highly reproducible screening results than the existing replicon system (in which a replicon-expressing vector is transiently transfected into cells).

### Example 7 Screening for Anti-Coronavirus Therapeutic Drug

### Example 7-1: Evaluation of Replicon Replication Level in Cell

VeroE6/Rep #3 cells were seeded to a 96-well plate at 6×10³ cells/well, and cultured overnight to adhere to the plate in a living state. On the following day, the culture medium of the 96-well plate was discarded and substituted with a culture medium containing each of the compounds of compound library 1 (Protein-Protein Interaction Inhibitor Library (manufactured by Selleck Co.): a library containing 177 compounds including inhibitors) and compound library 2 (Diversity Set (manufactured by Enamine Co.): a library containing 493 compounds having various structures), followed by culturing at 37° C. for 72 hours in the presence of 5% CO₂. The culture medium contained the each of the compounds at the final concentration of 10 μM and DMSO at the final concentration of 0.1%.

In order to evaluate the replicon replication level in the cells, a luciferase assay was performed using Renilla Luciferase Assay System (manufactured by Promega Corporation, #E2810). Specifically, the cell culture medium containing the each of the compounds was discarded, and then Renilla Luciferase Assay Lysis Buffer (manufactured by Promega Corporation), which had been diluted, was added to the wells at 20 μL/well, followed by incubation at room temperature for 30 minutes. Then, Renilla Luciferase Assay Substrate (manufactured by Promega Corporation), which had been diluted, was added to the wells at 100 μL/well. A GloMax (registered trademark) microplate luminometer (manufactured by Promega Corporation) was used to measure the luciferase activity. The each of the compounds was evaluated in four wells, and an average value was calculated. Regarding the compound observed that the luciferase activity decreased at the final concentration of 10 μM, the final concentration of the compound was decreased to perform the evaluation.

### Example 7-2: Evaluation of Cell Survival Rate

In the same manner as in Example 7-1, VeroE6/Rep #3 cells were cultured in a culture medium containing each of the compounds of compound library 1 and compound library 2.

In order to evaluate the cell survival rate after the treatment with the each of the compounds, CellTiter 96 (registered trademark) AQueous One Solution (manufactured by Promega Corporation) was used to perform an MTS assay. Specifically, the cell culture medium containing the each of the compounds was discarded and substituted with a culture medium containing an MTS reagent (CellTiter 96 (registered trademark) AQueous One Solution (manufactured by Promega Corporation)), followed by incubation at 37° C. for 2 hours in the presence of 5% CO₂. Then, a microplate reader (manufactured by Bio-Rad Laboratories, Inc.) was used to measure absorbance at a wavelength of 490 nm. The each of the compounds was evaluated in four wells, and an average value was calculated. Regarding the compound observed that the cell survival rate decreased at the final concentration of 10 μM, the final concentration of the compound was diluted to perform the evaluation.

Instead of the culture medium containing the each of the compounds, a culture medium containing coronavirus replication inhibitor EIDD-2801 (molnupiravir, manufactured by MedChemExpress Co., HY-135853) at the final concentration of 20 μM and DMSO at the final concentration of 0.1% was provided as a positive control, and a culture medium containing DMSO at the final concentration of 0.1% was provided as a negative control. In the same manners as in Example 7-1 and Example 7-2, the luciferase assay and the MTS assay were performed. Each of the positive control and the negative control was evaluated in four wells, and an average value was calculated.

Using the luciferase activity and the cell survival rate of the negative control group as standards, the replication inhibitory rate and the cell survival rate of each of the compound-treated groups were calculated.

Compared with the negative control group treated with DMSO, the luciferase activity, which is an index of the replicon replication level, significantly decreased in the positive control group treated with EIDD-2801. Meanwhile, a decrease in cell proliferation was not observed. This is considered to indicate that EIDD-2801 has an ability to inhibit the replicon proliferation.

Meanwhile, some of the groups treated with the library compounds involved significant decreases in the luciferase activity and the cell proliferation level. In order to distinguish the compounds that decrease the luciferase activity by the non-specific effect of such cytotoxicity from the compounds that significantly inhibit the replicon replication, the “effectiveness rate” of each of the compounds was calculated by standardizing the replicon replication inhibitory rate determined from the luciferase activity on the basis of the cell survival rate, according to Formula 1 below:

(Effectiveness rate of compound x)=(Replicon replication level)/(Cell survival rate) (Formula 1).

The effectiveness rate of EIDD-2801 was about 0.2.

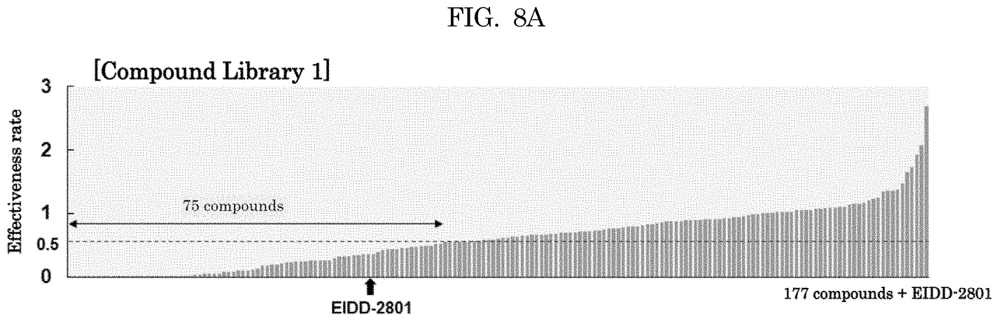

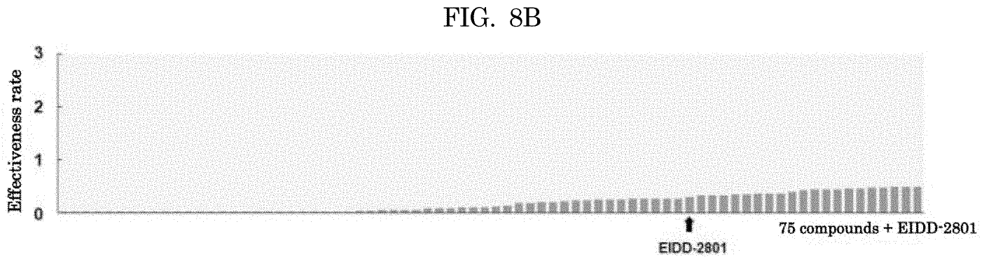

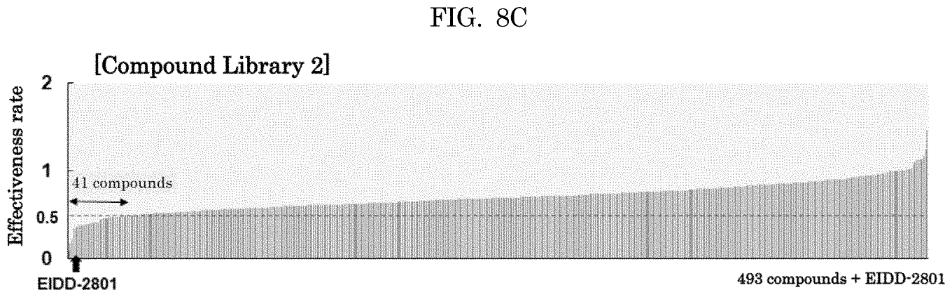

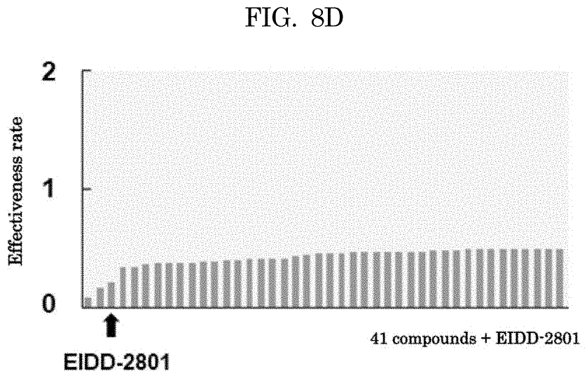

When the cutoff value of the effectiveness rate in the screening using VeroE6/Rep #3 was set to 0.5, as illustrated in FIG. 8A, 75 compounds of the 177 compounds in the compound library 1 had the effectiveness rate of lower than the cutoff value; i.e., 0.5, and 55 compounds thereof had the effectiveness rate of lower than that of EIDD-2801. FIG. 8B illustrates an enlarged view about the 75 compounds and EIDD-2801 in FIG. 8A. As illustrated in FIG. 8C, 41 compounds of the 493 compounds in the compound library 2 had the effectiveness rate of lower than the cutoff value; i.e., 0.5, and two compounds thereof had the effectiveness rate of lower than that of EIDD-2801. FIG. 8D is an enlarged view about the 41 compounds and EIDD-2801 in FIG. 8C.

Aspects of the present disclosure are as follows, for example.

<1> A cell line, which is of Accession No. NITE P-03458.

<2> A producing method of a cell line, the producing method including:

a transfection step of transfecting a vector into a cell, where the vector includes a nucleic acid sequence encoding a nonstructural protein of a coronavirus, a reporter protein including a drug-resistant protein, and a structural protein of the coronavirus; and

a selection step of performing selection on the cell obtained in the transfection step using a drug.

<3> The producing method according to <2> above, wherein the vector does not include all of the structural protein of the coronavirus.

<4> The producing method according to <2> or <3> above, wherein the reporter protein is a fusion protein of the drug-resistant protein and a luminescent protein or a fluorescent protein.

<5> The producing method according to any one of <2> to <4> above, wherein the vector is an artificial chromosome vector.

<6> The producing method according to any one of <2> to <5> above, wherein the coronavirus is SARS-CoV-2.

<7> The producing method according to <6> above, wherein the structural protein of the coronavirus includes an N structural protein of the SARS-CoV-2.

<8> The producing method according to any one of <2> to <7> above, wherein the cell is a VeroE6 cell.

<9> A vector, including:

a nucleic acid sequence encoding a nonstructural protein of a coronavirus, a reporter protein including a drug-resistant protein, and a structural protein of the coronavirus.

<10> An introduced cell, including:

the vector according to <9> above, which is introduced into the introduced cell.

<11> A screening method for an anti-coronavirus therapeutic drug, the screening method including:

using the introduced cell according to <10> above.

<12> A screening kit for an anti-coronavirus therapeutic drug, the screening kit including:

the introduced cell according to <10> above.

What is claimed is:

1. A cell line, which is of Accession No. NITE P-03458.

2. A producing method of a cell line, the producing method comprising:
transfecting a vector into a cell, where the vector includes a nucleic acid sequence encoding a nonstructural protein of a coronavirus, a reporter protein including a drug-resistant protein, and a structural protein of the coronavirus; and
performing selection on the cell obtained in the transfecting using a drug.

3. The producing method according to claim 2, wherein the vector does not include all of the structural protein of the coronavirus.

4. The producing method according to claim 2, wherein the reporter protein is a fusion protein of the drug-resistant protein and a luminescent protein or a fluorescent protein.

5. The producing method according to claim 2, wherein the vector is an artificial chromosome vector.

6. The producing method according to claim 2, wherein the coronavirus is SARS-CoV-2.

7. The producing method according to claim 6, wherein the structural protein of the coronavirus includes an N structural protein of the SARS-CoV-2.

8. The producing method according to claim 2, wherein the cell is a VeroE6 cell.

9. A vector, comprising:
a nucleic acid sequence encoding a nonstructural protein of a coronavirus, a reporter protein including a drug-resistant protein, and a structural protein of the coronavirus.

10. An introduced cell, comprising:
the vector according to claim 9, which is introduced into the introduced cell.

11. A screening method for an anti-coronavirus therapeutic drug, the screening method comprising:
using the introduced cell according to claim 10.

12. A screening kit for an anti-coronavirus therapeutic drug, the screening kit comprising:
the introduced cell according to claim 10.
